(19) United States
(12) Patent Application Publication
(10) Pub. No.: US 2007/0082919 A1
(43) Pub. Date: Apr. 12, 2007

(54) Process for Preparing a Co-Precipitate of a Non-Crystalline Solid Drug Substance

(75) Inventors: Roger Barrett, Stevenage (GB); Richard Anthony Ward, Stevenage (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Smithkline Beecham Corporation, Philadelphia, PA (US)

(21) Appl. No.: 10/575,208

(22) PCT Filed: Oct. 12, 2004
(86) PCT No.: PCT/US04/33521

§ 371 Date: Apr. 7, 2006

(30) Foreign Application Priority Data
Oct. 14, 2003 (GB) 0324086.8

(51) Int. Cl. A61K 31/522

(52) U.S. Cl. 514/263.36; 514/263.1

(57) ABSTRACT

The present invention provides processes for preparing co-precipitates of non-crystalline, solid drug substances, such as (E)-4-(1,3-bis(cyclohexylmethyl)-1,2,3,6 -tetrahydro-2,6-dioxo-9H-purin-8-yl)cinnamic acid nonaethylene glycol methyl ether ester and solvates thereof.

### BACKGROUND OF THE INVENTION

A co-precipitate is a type of solid dispersion of a drug substance which is produced by jointly precipitating from solution the drug substance together with a carrier, such as a polymer. Co-precipitates have been employed in the pharmaceutical industry to improve the oral bioavailability of sparingly soluble drug substances. Ohm, European J. Pharmaceutics and Biopharmaceutics 49:183-189 (2000). Nifedipine (ADALAT T10®) is an example of this approach. One method for forming a co-precipitate involves solubilizing both the drug substance and the carrier (e.g. polyvinylpyrrolidone (PVP)) in an organic solvent and then removing the solvent to cause the co-precipitation of the drug substance and carrier. This technique typically results in an intimate mixture of the drug substance and the carrier in the final product. Examples of co-precipitates in the pharmaceutical industry are described in EP Patent No. 828479 to Glaxo, PCT Publication No. WO02/038127 to Bradford, and PCT Publication No. WO01/03821 to Bradford.

While co-precipitates have been employed to improve oral bioavailability of drug substances, it is believed that no literature methods exist for co-precipitation techniques which can improve the physical or handling properties of drug substances which exhibit poor handling properties. For example, it would be desirable to provide a method for improving the physical properties of a non-particulate, non-crystalline solid drug substance, such as a wax-like drug substance, to provide the drug substance in the form of a free-flowing particulate material which is more conveniently handled using conventional pharmaceutical formulating techniques.

(E)4-(1,3-bis(cyclohexylmethyl)-1,2,3,6-tetrahydro-2,6-dioxo-9H-purin-8-yl)cinnamic acid nonaethylene glycol methyl ether ester, methods for its preparation and use and pharmaceutical formulations containing the same are disclosed in PCT Publication No. 98/35966, published 20 Aug. 1998 and U.S. Pat. No. 6,355,646 to Daluge et al. The compound is a waxy solid.

### BRIEF SUMMARY OF THE INVENTION

As a first aspect the present invention provides a process for preparing a particulate form of a non-crystalline, solid drug substance. The process comprises the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein the non-aqueous solvent and the anti-solvent are miscible; and
b) isolating the co-precipitate.

As a second aspect, the present invention provides a process for improving the physical properties of a non-particulate, solid drug substance. The process comprises slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein the non-aqueous solvent and the anti-solvent are miscible.

As a third aspect, the present invention provides a process for preparing a co-precipitate of a non-crystalline, solid drug substance. The process comprises slowly adding a co-precipitant solution comprising the drug substance and a co-precipitating excipient solubilized in a non-aqueous solvent, to a slurry comprising a core excipient dispersed in an anti-solvent, wherein the non-aqueous solvent and the anti-solvent are miscible.

As a fourth aspect, the present invention provides a pharmaceutical composition comprising a co-precipitate having a core and one or more drug layers distributed around the core. The core comprises core excipient. The drug layers comprise a drug substance and a co-precipitating excipient. The co-precipitate is prepared by slowly adding a co-precipitant solution comprising the drug substance and the co-precipitating excipient solubilized in a non-aqueous solvent, to a slurry comprising the core excipient dispersed in an anti-solvent, wherein the non-aqueous solvent and the anti-solvent are miscible.

As a fifth aspect, the present invention provides a pharmaceutical composition comprising a co-precipitate having a core comprising a core excipient and one or more drug layers distributed around the core wherein the drug layers comprise (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof and a co-precipitating excipient.

As a sixth aspect, the present invention provides a process for preparing an aqueous-based pharmaceutical formulation comprising a non-crystalline, solid drug substance having low solubility in aqueous media. The process comprises the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein the non-aqueous solvent and the anti-solvent are miscible;
b) isolating the co-precipitate; and
c) admixing the co-precipitate with a pharmaceutically acceptable aqueous media to provide an aqueous-based pharmaceutical formulation.

In another aspect, the present invention provides a solid particulate drug substance produced by the process comprising the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein the non-aqueous solvent and the anti-solvent are miscible; and
b) isolating the co-precipitate.

As another aspect, the present invention provides another process for preparing a particulate form of a non-crystalline, solid drug substance. The process comprises the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, to prepare a co-precipitate, wherein the non-aqueous solvent is miscible with the drug solvent and the anti-solvent; and
d) isolating the co-precipitate.

As another aspect, the present invention provides another process for improving the physical properties of a non-particulate, solid drug substance. The process comprises the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, to prepare a co-precipitate, wherein the non-aqueous solvent is miscible with the drug solvent and the anti-solvent.

As another aspect, the present invention provides another process for preparing a co-precipitate of a non-crystalline, solid drug substance. The process comprises the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, wherein the non-aqueous solvent is miscible with the drug solvent and the anti-solvent.

As another aspect, the present invention provides a pharmaceutical composition comprising a co-precipitate having a core comprising the drug substance and one or more co-precipitant layers distributed around the core, wherein the co-precipitant layers comprise a co-precipitating excipient, and wherein the co-precipitate is prepared by the process comprising the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, wherein the non-aqueous solvent is miscible with the drug solvent and the anti-solvent.

As another aspect, the present invention provides another pharmaceutical composition comprising a co-precipitate having a core comprising (E)4-(1,3-bis(cyclohexylmethyl)-1,2,3,6-tetrahydro-2,6-dioxo-9H-purin-8-yl)cinnamic acid nonaethylene glycol methyl ether ester or a solvate thereof and one or more co-precipitant layers distributed around said core.

As another aspect, the present invention provides another process for preparing an aqueous-based pharmaceutical formulation comprising a non-crystalline, solid drug substance having low solubility in aqueous media. The method comprises the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, to prepare a co-precipitate, wherein the non-aqueous solvent is miscible with the drug solvent and the anti-solvent;
d) isolating the co-precipitate; and
e) admixing the co-precipitate with a pharmaceutically acceptable aqueous media to provide an aqueous-based pharmaceutical formulation.

As another aspect, the present invention provides a solid particulate drug substance produced by a process comprising the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, to prepare a co-precipitate, wherein the non-aqueous solvent is miscible with the drug solvent and the anti-solvent; and
d) isolating the co-precipitate.
These and other aspects of the invention are described in further detail in the description and examples which follow.

### BRIEF DESCRIPTION OF THE DRAWINGS

FIG. 1 is an illustration of a co-precipitate particle produced according to a process of the invention.

FIG. 2 is an illustration of a co-precipitate particle produced according to a process of the invention.

### DETAILED DESCRIPTION OF THE INVENTION

The present invention provides processes for the preparation of a particulate form of a non-crystalline, solid drug substance. As such, the present invention provides methods for improving the physical properties, and particularly the handling, of a non-crystalline, solid drug substance by providing processes for converting the non-crystalline, solid drug substance into particulate form.

The processes of the present invention may be employed to prepare co-precipitates of a variety of non-crystalline, solid drug substances. In particular, the processes of the present invention are useful for converting tacky, or wax-like drug substances or amorphous drug substances into a more free-flowing, particulate form.

The processes of the present invention are particularly useful when the drug substance is a wax-like drug substance.

In one embodiment of the present invention, the drug substance is a compound of formula (I): 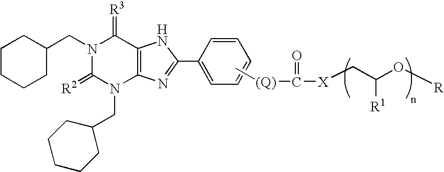
wherein:
X is —O—or —NH—;
Q is (—CH₂—)ₚ, (—CH═CH—)ₚ, or (—C≡C—) where p is an integer of from 1 to 4;
R¹ is H or methyl;
R² and R³ independently represent O or S;
n is an integer of 1 to 50;
and R is H or methyl
or a solvate thereof.

Specific examples of compounds of formula (I) include but are not limited to
(E)-4-(1,3-Bis(cyclohexylmethyl)-1,2,3,6-tetrahydro-2,6-dioxo-9H-purin-8-yl)cinnamic acid nonaethylene glycol methyl ether ester;
(E)-4-(1,3-Bis(cyclohexylmethyl)-1,2,3,6-tetrahydro-2,6-dioxo-9H-purin-8-yl)cinnamic acid decaethylene glycol methyl ether ester;
(E)-3-( 1,3-Bis(cyclohexylmethyl)-1,2,3,6-tetrahydro-2,6-dioxo-9H-purin-8-yl] cinnamic acid nonaethylene glycol methyl ether ester;
(E)-4-(1,3-Bis(cyclohexylmethyl)-1,2,3,6-tetrahydro-2,6-dioxo-9H-purin-8-yl)cinnamic acid nonaethylene glycol methyl ether amide;
(E)-4-(1,3-Bis(cyclohexylmethyl)-1,2,3,6-tetrahydro-2,6-dioxo-9H-purin-8-yl)benzoic acid nonaethylene glycol methyl ether ester;
(E)-4-(1,3-Bis(cyclohexylmethyl)-1,2,3,6-tetrahydro-2,6-dioxo-9H-purin-8-yl)cinnamic acid triethylene glycol methyl ether ester;
(E)-4( 1,3-Bis(cyclohexylmethyl)-1,2,3,6-tetrahydro-2,6-dioxo-9H-purin-8-yl) cinnamic acid polyethylene glycol (n=7.2) methyl ether ester;
(E)-4(1,3-Bis(cyclohexylmethyl)-1,2,3,6-tetrahydro-2,6-dioxo-9H-purin-8-yl) cinnamic acid tetraethylene glycol methyl ether ester;
(E)-4(1,3-Bis(cyclohexylmethyl)-1,2,3,6-tetrahydro-2,6-dioxo-9H-purin-8-yl) cinnamic acid pentaethylene glycol methyl ether ester;
(E)-4-(1,3-Bis(cyclohexylmethyl)-1,2,3,6-tetrahydro-2,6-dioxo-9H-purin-8-yl) cinnamic acid hexaethylene glycol methyl ether ester;
(E)-4-(1,3-Bis(cyclohexylmethyl)-1,2,3,6-tetrahydro-2,6-dioxo-9H-purin-8-yl) cinnamic acid heptaethylene glycol methyl ether ester;
(E)4-(1,3-Bis(cyclohexylmethyl)-1,2,3,6-tetrahydro-2,6-dioxo-9H-purin-8-yl) cinnamic acid octaethylene glycol methyl ether ester;
(E)4-(1,3-Bis(cyclohexylmethyl)-1,2,3,6-tetrahydro-2,6-dioxo-9H-purin-8-yl) cinnamic acid polyethylene glycol (n=11.7) methyl ether ester;
(E)-+(1,3-Bis(cyclohexylmethyl)-1,2,3,6-tetrahydro-2,6-dioxo-9H-purin-8-yl) cinnamic acid hexaethylene glycol ester;
(E)+(1,3-Bis(cyclohexylmethyl)-1,2,3,6-tetrahydro-2,6-dioxo-9H-purin-8-yl) cinnamic acid polyethylene glycol (n=23.9) methyl ether ester;
(E)-+(1,3-Bis(cyclohexylmethyl)-1,2,3,6-tetrahydro-2,6-dioxo-9H-purin-8-yl) cinnamic acid polyethylene glycol (n=41.5) methyl ether ester;
(E)4-(1,3-Bis(cyclohexylmethyl)-1,2,3,6-tetrahydro-2,6-dioxo-9H-purin-8-yl) cinnamic acid polyethylene glycol (n=15) methyl ether ester;
(E)+(1,3-Bis(cyclohexylmethyl)-1,2,3,6-tetrahydro-2,6-dioxo-9H-purin-8-yl) cinnamic acid polyethylene glycol (n=32.2) ester;
(E)-4-(1,3-Bis(cyclohexylmethyl)-1,2,3,6-tetrahydro-2,6-dioxo-9H-purin-8-yl) cinnamic acid polyethylene glycol (n=18.9) ester;
(E)-4(1,3-Bis(cyclohexylmethyl)-1,2,3,6-tetrahydro-2,6-dioxo-9H-purin-8-yl) cinnamic acid polyethylene glycol (n=13) ester;
(E)-3-(1,3-Bis(cyclohexylmethyl)-1,2,3,6-tetrahydro-2,6-dioxo-9H-purin-8-yl) benzoic acid nonaethylene glycol methyl ether ester;
(E)-2-(1,3-Bis(cyclohexylmethyl)-1,2,3,6-tetrahydro-2,6-dioxo-9H-purin-8-yl) benzoic acid nonaethylene glycol methyl ether ester;
(E)-4(1,3-Bis(cyclohexylmethyl)-1,2,3,6-tetrahydro-6-oxo-2-thioxo--9H-purin-8-yl) cinnamic acid nonaethylene glycol methyl ether ester;
   and solvates thereof.

In one particular embodiment of the present invention the drug substance is (E)-4-( 1,3-bis(cyclohexylmethyl)-1,2,3,6-tetrahydro-2,6-dioxo-9H-purin-8-yl)cinnamic acid nonaethylene glycol methyl ether ester or a solvate thereof.

The processes of the present invention are further advantageous for improving the stability of compounds which are susceptible to degradation by aerial oxidation. In particular, the compounds of formula (I) are known to be subject to degradation by aerial oxidation. The susceptibility to aerial oxidation of the compound is reduced by the co-precipitates prepared according to the present invention because of the intimate incorporation of the drug substance in the co-precipitate, which thereby reduces the surface area exposed to aerial oxidation.

The compounds of formula (I) may be prepared according to the processes described in U.S. Pat. No. 6,355,646 to Daluge, the subject matter of which is incorporated herein by reference in its entirety.

The drug substance may comprise two or more therapeutically effective agents in combination, provided that the agents are compatible (i.e., the agents are not deleterious to each other in the processes of the present invention).

Process 1

In one embodiment of the present invention, a particulate form of a non-crystalline, solid drug substance may be prepared by the process comprising the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein the non-aqueous solvent and the anti-solvent are miscible; and
b) isolating the co-precipitate.

Conveniently any conventional pharmaceutically acceptable excipient may be employed as the co-precipitating excipient, provided that it is compatible with the drug substance in solution. Suitable co-precipitating excipients include but are not limited to sugars, sugar alcohols, polymers, starches, salts and mixtures thereof. Particular examples of suitable co-precipitating excipients are selected from sorbitol, sucrose, glucose, fructose, lactose, xylitol, maltodextrin, hydroxypropylmethylcellulose (HPMC), polyvinylpyrrolidone, saccharin, starch 1500, sodium chloride and mixtures thereof. In one embodiment, the co-precipitating excipient is selected from sorbitol, sucrose, glucose, fructose, lactose, xylitol, maltodextrin and mixtures thereof. In one particular embodiment, the co-precipitating excipient is sorbitol.

The non-aqueous solvent into which the drug substance and the co-precipitating excipient are solubilized may include any suitable non-aqueous solvent In which both the drug substance and the co-precipitating excipient substantially completely dissolve. If necessary or desired, increased temperature may be employed to facilitate or expedite the solubilization of the drug substance and/or the co-precipitating excipient in the non-aqueous solvent. Examples of suitable non-aqueous solvents for use in the processes of the present invention include but are not limited to organic acids, alcohols, polar aprotic solvents, and mixtures thereof.

In one embodiment, the non-aqueous solvent is selected from acetic acid, propionic acid, formic acid, dimethylsulfoxide, dimethylformamide, ethanol, methanol and mixtures thereof. In one particular embodiment, the non-aqueous solvent is acetic acid.

In one embodiment of the present invention the co-precipitant solution further comprises an amount of drug substance stabilizer which is sufficient to increase the shelf-life of the drug substance in a pharmaceutical composition. Pharmaceutically acceptable drug substance stabilizers are known in the art and may be employed for this purpose. The choice of the particular drug substance stabilizer will depend upon the particular drug substance. In one embodiment, when the drug substance is a compound of formula (I) above, particularly when the drug substance is (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof, the drug substance stabilizer is butylated hydroxy toluene (BHT).

When the drug substance stabilizer is employed in the co-precipitant solution, it becomes incorporated into the resulting co-precipitate.

The amount of drug substance stabilizer to be incorporated will depend upon the particular drug substance and drug substance stabilizer chosen and will be apparent to those skilled in the art of pharmaceutical development. In one embodiment, the amount of drug substance stabilizer is from about 1 ppm to about 1000 ppm with respect to the drug substance. In one embodiment, the amount of drug substance stabilizer is from about 1 ppm to about 750 ppm with respect to the drug substance. In one particular embodiment, the drug substance stabilizer is present in an amount of from about 10 ppm to about 500 ppm with respect to the amount of drug substance.

The co-precipitant solution is then added slowly to a slurry comprising a core excipient dispersed in an anti-solvent. The core excipient may be any pharmaceutically acceptable, solid excipient. Examples of suitable core excipients include but are not limited to sugars, sugar alcohols, starches, salts and mixtures thereof. Particular examples of suitable core excipients are selected from sorbitol, sucrose, glucose, fructose, lactose, xylitol, maltodextrin, saccharin, sodium chloride and mixtures thereof. In one embodiment, the core excipient is sorbitol.

As used herein “anti-solvent” refers to a liquid in which the drug substance and the co-precipitating excipients are substantially completely insoluble. The anti-solvent should be chosen such that it is miscible with the non-aqueous solvent and allows robust recovery (>85%) of the co-precipitate. As used herein, the term “miscible” refers to the property of the solvents to dissolve in each other such that a single-phase solution results from the combination of the non-aqueous solvent and the anti-solvent. Suitable anti-solvents are known in the art of co-precipitation and include, for example alkane solvents. Specific examples of suitable anti-solvents include but are not limited to cyclohexane, isohexane, heptane, iso-octane and mixtures thereof. In one embodiment, the anti-solvent is iso-octane.

The amount of drug substance, co-precipitating excipient and core excipient which are employed to prepare the co-precipitate of the present invention will depend upon the particular drug substance and excipients selected. Generally, the amount of drug substance, co-precipitating excipient and core excipient will be sufficient to yield a co-precipitate (i.e., product) having a ratio of drug substance to combined excipients (i.e., co-precipitating excipient+core excipient) of from about 1:100 to about 50:50. In one embodiment, the ratio of drug substance to combined excipients (i.e., co-precipitating excipient+core excipient) in the co-precipitate product is from about 15:85 to about 40:60. In one embodiment, the ratio of drug substance to combined excipients (i.e., co-precipitating excipient +core excipient) is about 25:75.

Conveniently, the addition of the co-precipitant solution to the slurry may be carried out under ambient conditions. However, the addition may, if desired be carried out at reduced temperatures as well. The co-precipitant solution is added to the slurry slowly, optionally while stirring. In one embodiment, the co-precipitant solution is added drop-wise to the slurry, optionally while stirring.

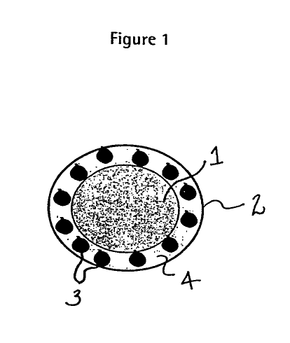

The addition of the co-precipitant solution to the slurry comprising the core excipient in the anti-solvent, results in the formation of a co-precipitate comprising the drug substance, the co-precipitating excipient and the core excipient. Without wishing to be bound by any particular theory, it is presently believed that the resultant co-precipitate, as illustrated in FIG. 1, comprises a core of excipient 1 having dispersed thereon one or more drug layers 2 comprised of drug substance 3 and co-precipitating excipient 4.

The one or more drug layers dispersed around the core are not typically uniform in thickness and may vary. The resulting co-precipitate exhibits improved physical properties, particularly in terms of handling. It is a finely divided material with flow properties resembling those of the co-precipitating excipient. More specifically, the resulting co-precipitate is a free-flowing particulate or powder. In the case of a wax-like drug substance, the resulting co-precipitate no longer exhibits the tacky, wax-like characteristics of the original drug substance. Thus, in another aspect, the present invention provides a process for improving the physical properties of a non-crystalline, solid drug substance.

According to one specific embodiment of the present invention, the process for preparing the co-precipitate comprises slowly adding a co-precipitant solution comprising (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof and sorbitol solubilized in acetic acid, to a slurry comprising sorbitol dispersed in isooctane, to prepare a co-precipitate.

In another embodiment, the present invention provides a solid particulate drug substance produced by a process comprising the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein the non-aqueous solvent and the anti-solvent are miscible; and
b) isolating the co-precipitate.
   Process 2

According to another embodiment, the co-precipitate may be produced by a second process, which comprises the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, to prepare a co-precipitate, wherein the non-aqueous solvent is miscible with the drug solvent and the anti-solvent; and
d) isolating the co-precipitate.

The choice of drug solvent will depend upon the particular drug substance chosen. Generally, the drug substance should be substantially completely soluble in the drug solvent. For example, up to about 1 g of the drug substance should solubilize in about 5-10 mL of the drug solvent. Heating may be employed to facilitate or expedite solubilization of the drug substance in the drug solvent. In addition, the drug solvent should be miscible with the anti-solvent. Specific examples of suitable drug solvents for drugs which possess low solubility in aqueous media (such as for example the compounds of formula (I)) include but are not limited to chlorinated solvents, polar solvents, polar aprotic solvents, alcohols and mixtures thereof. More specifically, suitable drug solvents include but are not limited to dichloromethane, ethylacetate, tetrahydrofuran, dimethylformamide, dimethylsulfoxide, methanol, ethanol, isopropanol and mixtures thereof. In one embodiment, the drug solvent is dichloromethane.

The drug solution may further comprise a drug substance stabilizer as described above.

Suitable anti-solvents are described above.

It is desirable to admix the drug solution and the anti-solvent while stirring to facilitate the formation of the drug suspension. The step of admixing the drug solution and the anti-solvent may conveniently be carried out at ambient temperature or pressure, but elevated temperature and/or pressure may also be employed. If desired, during the preparation of the drug substance suspension, the anti-solvent may be removed and replaced with fresh anti-solvent.

The excipient solution which is added to the drug suspension comprises a co-precipitating excipient solubilized in a non-aqueous solvent. Suitable co- precipitating excipients are described above. The non-aqueous solvent should be chosen so that it is miscible with both the drug solvent and the anti-solvent such that a combination of the three solvents results in a single-phase solution. Accordingly, the choice of each of the drug solvent, anti-solvent and non-aqueous solvent will depend upon each other. Suitable non-aqueous solvents for use in this process of the present invention are described above.

The amount of drug substance and co-precipitating excipient which are employed to prepare the co-precipitate of the present invention will depend upon the particular drug substance and excipient selected. Generally, the amount of drug substance and co-precipitating excipient will be sufficient to yield a co-precipitate (i.e., product) having a ratio of drug substance to co-precipitating excipient of from about 1:100 to about 50:50. In one embodiment, the ratio of drug substance to co-precipitating excipient in the co-precipitate product is from about 15:85 to about 40:60. In one embodiment, the ratio of drug substance to co-precipitating excipient is about 25:75.

Conveniently, the addition of the excipient solution to the drug suspension may be carried out under ambient conditions. However, the addition may, if desired be carried out at reduced temperatures as well. If necessary or desired the reaction may be carried out while stirring. The addition of the excipient solution to the drug suspension should be carried out slowly, and in one embodiment, the addition is drop-wise.

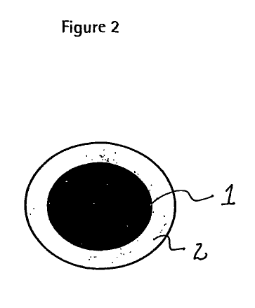

The addition of the excipient solution to the drug suspension, results in the formation of a co-precipitate comprising the drug substance and the co-precipitating excipient. Without wishing to be bound by any particular theory, it is presently believed that the resultant co-precipitate, as illustrated in FIG. 2, comprises a core of drug substance 1 having dispersed thereon one or more layers comprised of co-precipitating excipient 2. The layers of the co-precipitating excipient are not typically of uniform thickness and may vary. The resulting co-precipitate exhibits improved physical properties, particularly in terms of handling. It is a finely divided material with flow properties resembling those of the co-precipitating excipient. More specifically, the resulting co-precipitate is a free-flowing particulate or powder. In the case of a wax-like drug substance, the resulting co-precipitate no longer exhibits the tacky, wax-like characteristics of the original drug substance. Thus, in another aspect, the present invention provides another process for improving the physical properties of a non-crystalline, solid drug substance.

In one specific embodiment of the present invention, the process for preparing the co-precipitate comprises the steps of:
a) solubilizing (E)-4(1,3-bis(cyclohexylmethyl)-1,2,3,6-tetrahydro-2,6-dioxo-9H-purin-8-yl)cinnamic acid nonaethylene glycol methyl ether ester or a solvate thereof in dichloromethane to prepare a drug solution;
b) admixing the drug solution with iso-octane to prepare a drug suspension comprising (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof suspended in a mixture of dichloromethane and iso-octane; and
c) slowly adding to the drug suspension, an excipient solution comprising sorbitol solubilized in acetic acid, to prepare a co-precipitate.

In another embodiment, the present invention provides a solid particulate drug substance produced by a process comprising the steps of:
a) solubilizing the drug substance in a drug solvent to prepare a drug solution;
b) admixing the drug solution with an anti-solvent to prepare a drug suspension comprising the drug substance suspended in a mixture of the drug solvent and the anti-solvent, wherein the drug solvent and the anti-solvent are miscible;
c) slowly adding to the drug suspension, an excipient solution comprising a co-precipitating excipient solubilized in a non-aqueous solvent, to prepare a co-precipitate, wherein the non-aqueous solvent is miscible with the drug solvent and the anti-solvent; and
d) isolating the co-precipitate.

The co-precipitate produced according to either of the foregoing processes may be isolated by any suitable means of separating the co-precipitate from the mixture of non-aqueous solvent and anti-solvent. For example, the co-precipitate may be isolated by filtering. If filtering is employed, it may be desirable to subsequently dry the co-precipitate to remove any residual solvent or anti-solvent. The optional drying step may be carried out at ambient temperature and pressure or at elevated temperature and/or reduced pressure. In another embodiment, the co-precipitate is isolated by evaporating off the non-aqueous solvent and the anti-solvent.

The co-precipitate produced according to the foregoing processes may be used alone as a pharmaceutical composition or incorporated into a pharmaceutical composition comprising one or more pharmaceutically acceptable excipients or carriers. In one particular embodiment, the present invention provides a pharmaceutical composition comprising a co-precipitate having a core comprising a core excipient and one or more drug layers distributed around the core wherein the drug layers comprise (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof and a co-precipitating excipient.

In another particular embodiment, the pharmaceutical composition comprises a co-precipitate having a core comprising (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof and one or more co-precipitant layers distributed around the core.

Pharmaceutical compositions of the co-precipitates prepared according to the processes of the present invention may conveniently be prepared using conventional pharmaceutical formulation techniques. Possible formulations include those suitable for oral, sublingual, buccal, parenteral (for example subcutaneous, intramuscular, or intravenous), rectal, topical including transdermal, intranasal and inhalation administration. The most suitable means of administration for a particular patient will depend on the nature and severity of the condition being treated and on the nature of the active compound, with the optimum route of administration being within the discretion of the attendant physician.

Formulations suitable for oral administration may be provided as discrete units, such as tablets, capsules, cachets, lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically a flavoured base, such as sugar and acacia or tragacanth and pastilles comprising the active compound in an innert base, such as gelatine and glycerine or sucrose acacia.

Formulations suitable for parenteral administration typically comprise sterile aqueous solutions containing a predetermined concentration of the active compound; the solution is preferably isotonic with the blood of the intended recipient. Although such solutions are preferably administered intravenously, they may also be administered by subcutaneous or intramuscular injection.

Formulations suitable for rectal administration are preferably provided as unit-dose suppositories comprising the active ingredient in one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethyleneglycols, alcohols, and combinations thereof. The active ingredient is typically present in such formulations at a concentration of from 0.1 to 15% w/w.

Formulations of the invention may be prepared by any suitable method, typically by uniformly and intimately admixing the active compound with liquids or finely divided solid carriers or both, in the required proportions and then, if necessary, shaping the resulting mixture into the desired shape.

For example a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Suitable formulations for administration by inhalation include fine particle dusts or mists which may be generated by means of various types of metered dose pressurised aerosols, nebulisers, or insufflators.

For pulmonary administration via the mouth, the particle size of the powder or droplets is typically in the range 0.5 -10 μm, preferably 1-5 μm, to ensure delivery into the bronchial tree. For nasal administration, a particle size in the range 10-500 μm is preferred to ensure retention in the nasal cavity.

Metered dose inhalers are pressurized aerosol dispensers, typically containing a suspension or solution formulation of the active ingredient in a liquefied propellant. During use, these devices discharge the formulation through a valve adapted to deliver a metered volume, typically from 10 to 150 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol surfactants, such as oleic acid or sorbitan trioleate, anti-oxidants and suitable flavouring agents.

Nebulisers are commercially available devices that transform solutions or suspensions of the active ingredient into a therapeutic aerosol mist either by means of acceleration of a compressed gas typically air or oxygen, through a narrow venturi orifice, or by means of ultrasonic agitation. Suitable formulations for use in nebulisers consist of the active ingredient in a liquid carrier and comprising up to 40% w/w of the formulation, preferably less than 20% w/w. The carrier is typically water or a dilute aqueous alcoholic solution, preferably made isotonic with body fluids by the addition of, for example, sodium chloride. Optional additives include preservatives if the formulation is not prepared sterile, for example, methyl hydroxy-benzoate, anti-oxidants, flavouring agents, volatile oils, buffering agents and surfactants.

Suitable formulations for administration by insuffation include finely comminuted powders which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

The co-precipitates produced according to the processes of the present invention are particularly suited to preparing aqueous-based pharmaceutical formulations comprising a non-crystalline, solid drug substance having a low solubility in aqueous media. According to one embodiment of the invention, an aqueous-based pharmaceutical formulation of such a drug substance is prepared by a process comprising the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein the non-aqueous solvent and the anti-solvent are miscible;
b) isolating the co-precipitate; and
c) admixing the co-precipitate with a pharmaceutically acceptable aqueous media to provide an aqueous-based pharmaceutical formulation.

In another embodiment, the process for preparing an aqueous-based pharmaceutical formulation comprising a non-crystalline, solid drug substance having a low solubility in aqueous media comprises the steps of:
a) solubilizing the drug substance in a drug solvent to prepare a drug solution;
b) admixing the drug solution with an anti-solvent to prepare a drug suspension comprising the drug substance suspended in a mixture of drug solvent and anti-solvent, wherein the drug solvent and the anti-solvent are miscible;
c) slowly adding to the drug suspension, an excipient solution comprising a co-precipitating excipient solubilized in a non-aqueous solvent, to prepare a co-precipitate, wherein the non-aqueous solvent is miscible with the drug solvent and the anti-solvent;
d) isolating the co-precipitate; and
e) admixing the co-precipitate with a pharmaceutically acceptable aqueous media to provide an aqueous-based pharmaceutical formulation.

Suitable aqueous media include conventional pharmaceutically acceptable aqueous solvents such as water, saline, solutions of cyclodextrin in water, solutions of glucose in water and mixtures thereof. Such aqueous formulations may optionally further include one or more pH buffers and/or other conventional pharmaceutical additives.

The co-precipitates and pharmaceutical compositions containing the same which are prepared according to the processes of the present invention have use in medicinal therapy. In particular, the co-precipitates and pharmaceutical compositions wherein the drug substance is a compound of formula (I) are useful for the treatment of inflammatory conditions and immune disorders associated with the infiltration of leukocytes into inflamed tissue, and particularly for the treatment of those conditions described in U.S. Pat. No. 6,355,646, the subject matter of which is incorporated herein by reference in its entirety. In particular, such co-precipitates and compositions are useful for the treatment of inflammatory bowel disease, irritable bowel syndrome, functional dyspepsia, periodontal disease, eczema, rheumatoid arthritis and asthma.

Accordingly, the present invention, in another embodiment provides methods for the treatment of these conditions or diseases in a mammal, particularly a human. The methods comprise the step of administering a therapeutically effective amount of a co-precipitate according to the processes of the present invention. As used herein, the term “treatment” refers to alleviating the specified condition, eliminating or reducing the symptoms of the condition, slowing or eliminating the progression of the condition and preventing or delaying the re-occurrence of the condition in a previously afflicted subject.

The precise therapeutically effective amount of the co-precipitate will depend on a number of factors including, but not limited to, the drug substance and condition or disease being treated, the age and weight of the subject being treated, the severity of the condition or disease being treated, the nature of the formulation, and the route of administration, and will ultimately be at the discretion of the attendant physician or veternarian. Suitable doses of compounds of formula (I) will be apparent to those skilled in the art based upon the disclosure in U.S. Pat. No. 6,355,646.

The present invention is further illustrated by, but is not limited to, the following examples. In the following examples, “Compound 1” refers to (E)-4-(1,3-bis(cyclohexylmethyl)-1,2,3,6-tetrahydro-2,6-dioxo-9H-purin-8-yl)cinnamic acid nonaethylene glycol methyl ether ester; and “iso-octane” is also known as 2,2,4-trimethylpentane.

### EXAMPLE 1

Co-Precipitation with Sorbitol 1

A warm (30-40° C.) solution of Compound 1 (2.0 g, 1 wt) and Sorbitol (3 g, 1.5 wt) in acetic acid (12 ml, 6 vol) is added slowly over 20 min to a stirred mixture of Sorbitol (3 g, 1.5 wt) and iso-octane (150 ml, 75 vol) at room temperature (20-25° C.). The resulting mixture is stirred at room temperature for 30 min and the solids isolated by filtration. The solid product is washed with iso-octane (3×10 ml, 3×5 vol) and dried in vacuo at 40° C. Recovery: 7.5 g, 3.75 wt.

Analysis: Analysis showed 22% w/w loading of Compound on Sorbitol. (ie: 1 g of Compound 1 is contained in 4.48 g of co-precipitate).

### EXAMPLE 2

Co-Precipitation with Sorbitol 2

A warm (30-40° C.) solution of Compound 1(3 g, 1 wt) and Sorbitol (3 g, 1 wt) in acetic acid (12 ml, 4 vol) is added slowly over 20 min to a stirred mixture of Sorbitol (3 g, 1 wt) and iso-octane (200 ml, 67 vol) at room temperature (20-25° C.). The resulting mixture is stirred at room temperature for 30 min and the solids isolated by filtration. The solid product is washed with iso-octane (2×15 ml, 2×5 vol) and dried in vacuo at 40° C. Recovery: 8.4 g, 2.8 wt.

Analysis: Analysis showed 32% w/w loading of Compound 1 on Sorbitol. (ie: 1 g of Compound 1 is contained in 3.16 g of co-precipitate).

### EXAMPLE 3

Co-Precipitation with Xylitol 1

A warm (30-40° C.) solution of Compound 1 (1 g, 1 wt) and Xylitol (1.5 g, 1.5 wt) in acetic acid (6 ml, 6 vol) is added slowly to a stirred mixture of Xylitol (1.5 g, 1.5 wt) and iso-octane (75 ml, 75 vol) at room temperature (20-25° C.). The resulting mixture is stirred at room temperature for 20 min and the solids isolated by filtration. The solid product is washed with iso-octane (2×15ml, 2×15 vol) and dried In vacuo at 40° C. Recovery: 3.7 g, 3.7 wt.

Analysis: The product was a finely-divided solid which was observed to be slightly more “sticky” than the analogous product with Sorbitol.

### EXAMPLE 4

Co-Precipitation with Xylitol 2

A warm (85-90° C.) solution of Compound 1 (1g, 1 wt) and Xylitol (1.5 g, 1.5 wt) in denatured ethanol (IMS, 7.5 ml, 7.5 vol) is added slowly to a stirred mixture of Xylitol (1.5 g, 1.5 wt) and iso-octane (75 ml, 75 vol) at room temperature (20-25° C.). The resulting mixture is stirred at room temperature for 30 min and the solids isolated by filtration. The solid product is washed with iso octane (2×25 ml, 2×25 vol) and dried in vacuo at 40° C. Recovery: 3.7 g, 3.7 wt.

Analysis: This yielded a product very similar to Example 3.

### EXAMPLE 5

Co-Precipitation with Sorbitol and Maltodextrin

A warm (30-40° C.) solution of Compound 1 (1 g, 1 wt) and Sorbitol (1.5 g, 1.5 wt) in acetic acid (7.5 ml, 7.5 vol) is added slowly over 20 min to a stirred mixture of maltodextrin (1.5 g, 1.5 wt) and iso-octane (75 ml, 75 vol) at room temperature (20-25° C.). The resulting mixture is stirred at room temperature for 30 min and the solids isolated by filtration. The solid product is washed with iso-octane (2×10 ml, 2×10 vol) and dried in vacuo at 40° C. Recovery: 3.7 g, 3.7 wt.

Analysis: Analysis showed 22% w/w loading of Compound 1 on Sorbitol/maltodextrin. (ie: 1 g of Compound 1 is contained in 4.5 g of co-precipitate).

### EXAMPLE 6

Co-Precipitation with Xylitol and Maltodextrin

A warm (30-40° C.) solution of Compound 1 (1 g, 1 wt) and Xylitol (1.5 g, 1.5 wt) in acetic acid (7.5 ml, 7.5 vol) is added slowly over 20 min to a stirred mixture of maltodextrin (1.5 g, 1.5 wt) and iso-octane (75 ml, 75 vol) at room temperature (20-25° C.). The resulting mixture is stirred at room temperature for 30 min and the solids isolated by filtration. The solid product is washed with iso-octane (2×10 ml, 2×10 vol) and dried in vacuo at 40° C. Recovery: 3.7 g, 3.7 wt.

Analysis: Analysis showed 22% w/w loading of Compound 1 on Xylitol / maltodextrin. (ie: 1 g of Compound 1 is contained in 4.5 g of co-precipitate).

### EXAMPLE 7

Co-Precipitation with Sorbitol and Xylitol

A warm (30-40° C.) solution of Compound 1 (1 g, 1 wt) and Sorbitol (1.5 g, 1.5 wt) in acetic acid (7 ml, 7 vol) is added slowly over 20 min to a stirred mixture of Xylitol (1.5 g, 1.5 wt) and iso-octane (75 ml, 75vol) at room temperature (20-25° C.). The resulting mixture is stirred at room temperature for 30 min and the solids isolated by filtration. The solid product is washed with iso-octane (2×25 ml, 2×25 vol) and dried in vacuo at 40° C. Recovery: 3.6 g, 3.6 wt.

Analysis: Physical properties are very similar to Sorbitol co-precipitates of Examples 1 and 2.

### EXAMPLE 8

Co-Precipitation with Saccharin

A solution of Compound 1 (1 g, 1 wt) and Saccharin (1.5 g, 1.5 wt) in tetrahydrofuran (7.5 ml, 7.5 vol) is added slowly over 20 min to a stirred mixture of Saccharin (1.5 g, 1.5 wt) and iso-octane (100 ml, 100 vol) at room temperature (20-25° C.). The resulting mixture is stirred at room temperature for 30 min and the solids isolated by filtration. The solid product is washed with iso-octane (2×25 ml, 2×25 vol) and dried in vacuo at 40° C. Recovery: 3.6 g, 3.6 wt.

Analysis: Analysis showed 25% w/w loading of Compound 1 on Saccharin.

### EXAMPLE 9

Co-Precipitate with Sorbitol and Sodium Chloride

A warm (30-40° C.) solution of Compound 1 (1 g, 1 wt) and Sorbitol (1.5 g, 1.5 wt) in acetic acid (7 ml, 7 vol) is added slowly over 20 min to a stirred mixture of sodium chloride (1.5 g, 1.5 wt) and iso-octane (75 ml, 75 vol) at room temperature (20-25° C.). During the addition the co-precipitate starts to agglomerate so the iso-octane is decanted off and replaced with fresh iso-octane. The resulting mixture is stirred at room temperature for 30min and the solids isolated by filtration. The solid product is washed with iso-octane (2×25 ml, 2×25 vol) and dried in vacuo at 40° C. Recovery: 2.9 g, 2.9 wt.

Analysis: Analysis showed 19% w/w loading of Compound 1 on Sorbitol / sodium chloride.

### EXAMPLE 10

Co-Precipitation with Sorbitol 3

A warm (30-40° C.) solution of Compound 1 (0.5 g, 1 wt) and Sorbitol (0.75 g, 1.5 wt) in acetic acid (4 ml, 8 vol) is added slowly over 20 min to a stirred mixture of Sorbitol (1.5 g, 3 wt) in Heptane (75 ml, 150 vol) at room temperature (20-25° C.). The resulting mixture is stirred at room temperature for 30 min and the solids isolated by filtration. The solid product is washed with Heptane (2×25 ml, 2×50 vol) and dried in vacuo at 40° C. Recovery: 2.6 g, 2.6 wt.

Analysis: Analysis showed 16% w/w loading of Compound 1 on Sorbitol.

### EXAMPLE 11

Co-Precipitation with Sorbitol at Reduced Temperature

A warm (30-40° C.) solution of Compound 1 (1 g, 1 wt) and Sorbitol (1.5 g, 1.5 wt) in acetic acid (7.5 ml, 7.5 vol) is added slowly over 20 min to a stirred mixture of Sorbitol (1.5 g, 3 wt) in iso-octane (75 ml, 75 vol) at 0 to -3° C. The resulting mixture is stirred at 0 to -3° C. for 30 min and the solids isolated by filtration. The solid product is washed with iso-octane (2×25 ml, 2×25 vol) and dried in vacuo at 40° C. Recovery: 3.9 g, 3.9 wt.

Analysis: Analysis showed 22% w/w loading of Compound 1 on Sorbitol.

### EXAMPLE 12

Co-Precipitation Process 2

A solution of Compound 1 (5.3 g, 1 wt) in a mixture of dichloromethane (10 ml, 1.9 vol) and 2 -propanol (5 ml, 0.9 vol) is added slowly over 20 min to stirred iso-octane (300 ml, 57 vol) at room temperature. A solution of Sorbitol (15 g, 2.8 wt) in acetic acid (30 ml, 5.7 vol) is then added slowly over 20 min at room temperature. The resulting mixture is stirred at room temperature for 30 min and the solids isolated by filtration. The solid product is washed with iso-octane (3×10 ml, 3×1.9 vol) and dried in vacuo at 45° C. Recovery: 17.9 g, 3.4 wt.

Analysis: Microscopic analysis revealed Compound 1 coated with Sorbitol.

### EXAMPLE 13

Co-Precipitation Process 2

A solution of Compound 1 (5 g, 1 wt) in a mixture of dichloromethane (10 ml, 2 vol) and 2-propanol (5 ml, 1 vol) is added slowly over 20min to stirred iso-octane (400 ml, 80 vol) at room temperature. A solution of sorbitol (25 g, 5 wt) in acetic acid (50 ml, 10 vol) is then added slowly over 20 min at room temperature. The resulting mixture is stirred at room temperature for 30 min and the solids isolated by filtration. The solid product is washed with iso-octane (3×10 ml, 3×1.9 vol) and dried in vacua at 45° C.

### EXAMPLE 14

Co-Precipitation Process 2 with Xylitol.

A solution of Compound 1 (5 g, 1 wt) in a mixture of dichloromethane (10 ml, 2 vol) and 2-propanol (5 ml, 1 vol) is added slowly over 20 min to stirred iso-octane (300 ml, 60 vol) at room temperature. A solution of xylitol (15 g, 3 wt) in acetic acid (30 ml, 6 vol) is then added slowly over 20 min at room temperature. The resulting mixture is stirred at room temperature for 30 min and the solids isolated by filtration. The solid product is washed with iso-octane (3×10 ml, 3×1.9 vol) and dried in vacua at 45° C.

### EXAMPLE 15

Co-Precipitation Process 2

A solution of Compound 1 (5 g, 1 wt) in a mixture of dichloromethane (10 ml, 2 vol) and 2-propanol (5 ml, 1 vol) is added slowly over 20 min to stirred heptane (300 ml, 60 vol) at room temperature. A solution of sorbitol (15 g, 3 wt) in acetic acid (30 ml, 6 vol) is then added slowly over 20 min at room temperature. The resulting mixture is stirred at room temperature for 30 min and the solids isolated by filtration. The solid product is washed with heptane (3×10 ml, 3×1.9 vol) and dried in vacuo at 45° C.

### EXAMPLE 16

Co-Precipitation Process 2 with BHT

A solution of Compound 1 (5 g, 1 wt) in a mixture of dichloromethane (10 ml, 2 vol) and 2-propanol (5 ml, 1 vol) containing butylated hydroxy-toluene (BHT) (1 g, 0.2 wt) is added slowly over 20 min to stirred iso-octane (300 ml, 60 vol) at room temperature. A solution of sorbitol (15 g, 3 wt) in acetic acid (30 ml, 6 vol) is then added slowly over 20 min at room temperature. The resulting mixture is stirred at room temperature for 30 min and the solids isolated by filtration. The solid product is washed with iso-octane (3×10 ml, 3×1.9 vol) and dried in vacua at 45° C.

### EXAMPLE 17

Suspension Formulations

Suspensions containing Compound 1 Co-precipitate as prepared according to any of Examples 1-16, are comprised of the following functional ingredients in the typical ranges indicated:

General Suspension Formulation Guidelines

| | | Quantity |
|---|---|---|
| Function | Component | % w/w |
| Active | Compound 1 Coprecipitate | 4-20* |
| Suspending agent | Xanthan gum, Hydroxypropyl- | 0.1-5 |
| | methyl cellulose, microcrys- | |
| | talline cellulose and | |
| | carboxymethylcellulose sodium | |
| Preservative | Sodium benzoate, methyl/propyl | 0.1-5 |
| | parabens | |
| Wetting agent | Sodium lauryl sulphate, Tween | 0.1-2 |
| Antifoaming agent | Simethicone, Dimethicone | 0.2-5 |
| Buffering agent | Sodium citrate, sodium | 0.05-0.5 |
| | phosphate, sodium acetate | |
| Flavours | Lemon, orange etc | 0.01-0.1 |
| Solvent Vehicle | Water | 60-100 |
| Cosolvent | Glycerol, proylene glycol | 5-40 |
| vehicle | | |

| | | |
|---|---|---|
| | | Quantity |
| Function | Component | (% w/w) |

Suspension Formulation 1

| | | |
|---|---|---|
| Active ingredient | Compound 1/Sorbitol co- | 10.0* |
| | precipitate | |
| Anti-foaming | Simethicone 30% emulsion | 0.2-5.0 |
| agent | | |
| Preservative | Sodium benzoate | 0.02-0.50 |
| Flavouring agent | Lemon flavour | 0.01-0.10 |
| Vehicle | Citrate buffer, pH 4 | to 100 |

Suspension Formulation 2

| | | |
|---|---|---|
| Active ingredient | Compound 1/Sorbitol co- | 10.0* |
| | precipitate | |
| Suspending agent | Xanthan gum | 0.1-1.0 |
| Anti-foaming | Simethicone 30% emulsion, | 0.2-5.0 |
| agent | | |
| Preservative | Sodium benzoate, parabens | 0.02-0.50 |
| Flavouring agent | Lemon flavour | 0.01-0.10 |
| Vehicle | Citrate buffer, pH 4 | to 100 |

Suspension Formulation 3

| | | |
|---|---|---|
| Active ingredient | Compound 1/Sorbitol co- | 10.0* |
| | precipitate | |
| Wetting agent | Sodium lauryl sulphate | 0.1-2.0 |
| Suspending agent | Xanthan gum | 0.1-1.0 |
| Anti-foaming | Simethicone 30% emulsion | 0.2-5.0 |
| agent | | |
| Preservative | Sodium benzoate | 0.02-0.50 |
| Flavouring agent | Lemon flavour | 0.01-0.10 |
| Vehicle | Citrate buffer, pH 4 | to 100 |

*assumes drug/sorbitol ratio 1:3, equivalent to an API suspension strength of 2.5% w/w.

1. A process for preparing a particulate form of a non-crystalline, solid drug substance comprising the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein said non-aqueous solvent and said anti-solvent are miscible;
b) isolating said co-precipitate.

2. The process according to claim 1, wherein said drug substance is (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof.

3. The process according to claim 1, wherein said co-precipitating excipient is selected from sorbitol, sucrose, glucose, fructose, lactose, xylitol, maltodextrin, hydroxypropylmethylcellulose, polyvinylpyrrolidone, starch 1500, sodium chloride and mixtures thereof.

4. The process according to claim 1, wherein said non-aqueous solvent is selected from organic acids, alcohols, polar aprotic solvents, and mixtures thereof.

5. The process according to claim 1, wherein said core excipient is selected from sorbitol, sucrose, glucose, fructose, lactose, xylitol, maltodextrin and mixtures thereof.

6. The process according to claim 1, wherein said anti-solvent is an alkane solvent.

7. A process for preparing a co-precipitate of a non-crystalline, solid drug substance comprising slowly adding a co-precipitant solution comprising the drug substance and a co-precipitating excipient solubilized in a non-aqueous solvent, to a slurry comprising a core excipient dispersed in an anti-solvent, wherein said non-aqueous solvent and said anti-solvent are miscible.

8. A pharmaceutical composition comprising a co-precipitate having a core comprising core excipient and one or more drug layers distributed around said core, wherein said drug layers comprise a drug substance and a co-precipitating excipient, and wherein said co-precipitate is prepared by slowly adding a co-precipitant solution comprising said drug substance and said co-precipitating excipient solubilized in a non-aqueous solvent, to a slurry comprising said core excipient dispersed in an anti-solvent, wherein said non-aqueous solvent and said anti-solvent are miscible.

9. A pharmaceutical composition comprising a co-precipitate having a core comprising a core excipient and one or more drug layers distributed around said core wherein said drug layers comprise (E)-4-(1,3-bis(cyclohexylmethyl)-1 ,2,3,6-tetrahydro-2,6-dioxo-9H-purin-8-yl)cinnamic acid nonaethylene glycol methyl ether ester or a solvate thereof and a co-precipitating excipient.

10. A process for preparing an aqueous-based pharmaceutical formulation comprising a non-crystalline, solid drug substance having low solubility in aqueous media, said process comprising the steps of:
a) slowly adding a co-precipitant solution comprising the drug substance and a co-precipitating excipient solubilized in a non-aqueous solvent, to a slurry comprising a core excipient dispersed in an anti-solvent, to prepare a co-precipitate, wherein said non-aqueous solvent and said anti-solvent are miscible;
b) isolating said co-precipitate; and
c) admixing said co-precipitate with a pharmaceutically acceptable aqueous media to provide an aqueous-based pharmaceutical formulation.

11. A process for preparing a particulate form of a non-crystalline, solid drug substance comprising the steps of:
a) solubilizing said drug substance in a drug solvent to prepare a drug solution;
b) admixing said drug solution with an anti-solvent to prepare a drug suspension comprising said drug substance suspended in a mixture of said drug solvent and said anti-solvent, wherein said drug solvent and said anti-solvent are miscible;
c) slowly adding to said drug suspension, an excipient solution comprising a co-precipitating excipient solubilized in a non-aqueous solvent, to prepare a co-precipitate, wherein said non-aqueous solvent is miscible with said drug solvent and said anti-solvent; and
d) isolating said co-precipitate.

12. The process according to claim 11, wherein said drug substance is (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof.

13. The process according to claim 11, wherein said drug solvent is selected from dichloromethane, ethylacetate, tetrahydrofuran, dimethylformamide, dimethylsulfoxide, methanol, ethanol, isopropanol and mixtures thereof.

14. The process according to claim 11, wherein said anti-solvent is an alkane solvent.

15. The process according to claim 11, wherein said co-precipitating excipient is selected from sorbitol, sucrose, glucose, fructose, lactose, xylitol, maltodextrin, hydroxypropylmethylcellulose, polyvinylpyrrolidone, starch 1500, sodium chloride and mixtures thereof.

16. The process according to claim 11, wherein said non-aqueous solvent is selected from organic acids, alcohols, polar aprotic solvents, and mixtures thereof.

17. A process for preparing a co-precipitate of a non-crystalline, solid drug substance comprising the steps of:
a) solubilizing said drug substance in a drug solvent to prepare a drug solution;
b) admixing said drug solution with an anti-solvent to prepare a drug suspension comprising said drug substance suspended in a mixture of said drug solvent and said anti-solvent, wherein said drug solvent and said anti-solvent are miscible;
c) slowly adding to said drug suspension, an excipient solution comprising a co-precipitating excipient solubilized in a non-aqueous solvent, wherein said non-aqueous solvent is miscible with said drug solvent and said anti-solvent.

18. A pharmaceutical composition comprising a co-precipitate having a core comprising said drug substance and one or more co-precipitant layers distributed around said core, wherein said co-precipitant layers comprise a co-precipitating excipient, and wherein said co-precipitate is prepared by the process comprising the steps of:
a) solubilizing said drug substance in a drug solvent to prepare a drug solution;
b) admixing said drug solution with an anti-solvent to prepare a drug suspension comprising said drug substance suspended in a mixture of said drug solvent and said anti-solvent, wherein said drug solvent and said anti-solvent are miscible;
c) slowly adding to said drug suspension, an excipient solution comprising a co-precipitating excipient solubilized in a non-aqueous solvent, wherein said non-aqueous solvent is miscible with said drug solvent and said anti-solvent.

19. A pharmaceutical composition comprising a co-precipitate having a core comprising (E)-4-(1,3-bis(cyclohexylmethyl)-1,2,3,6-tetrahydro-2,6-dioxo-9H-purin-8-yl)cinnamic acid nonaethylene glycol methyl ether ester or a solvate thereof and one or more co-precipitant layers distributed around said core.

20. A process for preparing an aqueous-based pharmaceutical formulation comprising a non-crystalline, solid drug substance having low solubility in aqueous media, said process comprising the steps of:
a) solubilizing said drug substance in a drug solvent to prepare a drug solution;
b) admixing said drug solution with an anti-solvent to prepare a drug suspension comprising said drug substance suspended in a mixture of said drug solvent and said anti-solvent, wherein said drug solvent and said anti-solvent are miscible;
c) slowly adding to said drug suspension, an excipient solution comprising a co-precipitating excipient solubilized in a non-aqueous solvent, to prepare a co-precipitate, wherein said non-aqueous solvent is miscible with said drug solvent and said anti-solvent;
d) isolating said co-precipitate; and
e) admixing said co-precipitate with a pharmaceutically acceptable aqueous media to provide an aqueous-based pharmaceutical formulation.
